(19) United States
(12) Patent Application Publication
(10) Pub. No.: US 2026/0026887 A1
(43) Pub. Date: Jan. 29, 2026

(54) SHAPE LOCALIZED FLEXIBLE INSTRUMENT

(71) Applicant: Intuitive Surgical Operations, Inc., Sunnyvale, CA (US)

(72) Inventors: Troy K. Adebar, Sunnyvale, CA (US); Federico Barbagli, Sunnyvale, CA (US)

(73) Assignee: Intuitive Surgical Operations, Inc., Sunnyvale, CA (US)

(21) Appl. No.: 18/997,303

(22) PCT Filed: Jul. 19, 2023
(86) PCT No.: PCT/US2023/028102

§ 371 Date: Jan. 21, 2025

Related U.S. Application Data
(60) Provisional application No. 63/391,123, filed on Jul. 21, 2022.

(51) Int. Cl. A61B 34/20; A61B 1/005; A61B 1/267; A61B 17/00; A61B 34/30

(52) U.S. Cl. CPC A61B 34/20; A61B 1/009; A61B 1/267; A61B 17/00234; A61B 34/30; A61B 2017/00809; A61B 2034/2051; A61B 2034/2061; A61B 2034/301

(57) ABSTRACT

A medical tracking system may include a flexible elongate device, a shape sensor configured to measure a shape of the flexible elongate device, and a controller including at least one processor. The controller may be configured to register a reference shape of the flexible elongate device to an anatomical reference frame, identify, using the shape sensor, the reference shape at a first portion of the flexible elongate device, determine, using the shape sensor, a spatial relationship between a second portion of the flexible elongate device and the first portion and determine a position of the second portion of the flexible elongate device in the anatomical reference frame based on the registration of the reference shape to the anatomical reference frame and the spatial relationship between the first portion and the second portion of the flexible elongate device.

### CROSS-REFERENCE TO RELATED APPLICATIONS

This application claims the benefit of priority under 35 U S.C. § 119(e) to U.S. Provisional Patent Application Ser. No. 63/391,123, filed on Jul. 21, 2022, which is hereby incorporated by reference herein in its entirety.

### FIELD

Disclosed embodiments are related to shape localized flexible instruments and related methods of use.

### BACKGROUND

Minimally invasive medical techniques are intended to reduce the amount of tissue that is damaged during medical procedures, thereby reducing patient recovery time, discomfort, and deleterious side effects. Such minimally invasive techniques may be performed through natural orifices in a patient anatomy or through one or more surgical incisions. Through these natural orifices or incisions clinicians may insert minimally invasive medical instruments (including surgical, diagnostic, therapeutic, or biopsy instruments) to reach a target tissue location. To assist with reaching the target tissue location, the location and movement of the medical instruments may be correlated with pre-operative or intra-operative images of the patient anatomy. With the image-guided instruments correlated to the images, the instruments may navigate natural or surgically created passageways in anatomic systems such as the lungs, the colon, the intestines, the kidneys, the heart, the circulatory system, or the like. Traditional instrument tracking and referencing systems may require the use of a computer-assisted system (e.g., a robotic system) that operates within a known three-dimensional space. Some conventional computer-assisted systems may employ a shape sensor which provides information regarding the three-dimensional shape of a portion of a flexible computer-assisted system.

### SUMMARY

In some embodiments, a medical tracking system comprises a flexible elongate device, a shape sensor configured to measure a shape of the flexible elongate device, and a controller comprising at least one processor. The controller is configured to register a reference shape of the flexible elongate device to an anatomical reference frame. The controller is also configured to identify, using the shape sensor, the reference shape at a first portion of the flexible elongate device. The controller is also configured to determine, using the shape sensor, a spatial relationship between a second portion of the flexible elongate device and the first portion. The controller is also configured to determine a position of the second portion of the flexible elongate device in the anatomical reference frame based on the registration of the reference shape to the anatomical reference frame and the spatial relationship between the first portion and the second portion of the flexible elongate device.

In some embodiments, a method of tracking a medical system comprising a flexible elongate device includes the following. The method comprises registering a reference shape of the flexible elongate device to an anatomical reference frame. The method also comprises identifying, using a shape sensor, the reference shape at a first portion of the flexible elongate device. The method also comprises determining, using the shape sensor, a spatial relationship between a second portion of the flexible elongate device and the first portion. The method also comprises determining a position of the second portion of the flexible elongate device in the anatomical reference frame based on the registration of the reference shape to the anatomical reference frame and the spatial relationship between the first portion and the second portion of the flexible elongate device.

In some embodiments, a non-transitory computer-readable storage medium stores instructions that, when executed by at least one processor associated with a computer-assisted device, causes the at least one processor to perform a method. The method comprises registering a reference shape of the flexible elongate device to an anatomical reference frame. The method also comprises identifying, using a shape sensor, the reference shape at a first portion of the flexible elongate device. The method also comprises determining, using the shape sensor, a spatial relationship between a second portion of the flexible elongate device and the first portion. The method also comprises determining a position of the second portion of the flexible elongate device in the anatomical reference frame based on the registration of the reference shape to the anatomical reference frame and the spatial relationship between the first portion and the second portion of the flexible elongate device.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a computer-assisted system, in accordance with embodiments of the present disclosure.

FIG. 2 illustrates a flexible elongate device utilizing aspects of the present disclosure.

FIG. 3 illustrates the distal end of the flexible elongate device of FIG. 2 positioned within a human lung.

FIG. 4 depicts a flexible computer-assisted system and a flexible manual device in accordance with embodiments of the present disclosure.

FIG. 5 depicts a schematic of a flexible computer-assisted system and a flexible manual device disposed in a lumen in accordance with embodiments of the present disclosure.

FIG. 6A depicts a schematic of a flexible manual device in a first state disposed in a lumen in accordance with embodiments of the present disclosure.

FIG. 6B depicts a schematic of a flexible manual device in a second state disposed in a lumen in accordance with embodiments of the present disclosure.

FIG. 7 is a flow chart for an embodiment of tracking a medical system comprising a flexible elongate device.

FIG. 8 depicts a schematic of a flexible computer-assisted system and a flexible manual device disposed in a lumen in accordance with embodiments of the present disclosure.

FIG. 9 depicts a schematic of a flexible manual device disposed in an adapter in accordance with embodiments of the present disclosure.

### DETAILED DESCRIPTION

Instrument tracking and referencing systems may make use of a flexible computer-assisted system (e.g., a robotic system) that operates within a known three-dimensional space. Control of one or more actuators of a flexible computer-assisted system may allow the computer assisted system to control the articulation (e.g., pitch and yaw) and insertion or retraction of a flexible elongate device. Use of the one or more actuators may allow the flexible computer-assisted system to maintain knowledge of a pose of one or more portions of the flexible computer-assisted system (e.g., an instrument, a distal end of a flexible elongate device, etc.) as the pose changes based on the commands to the one or more actuators. Additionally, a flexible computer-assisted system may employ one or more sensors (e.g., shape sensors) that provide information regarding the pose of one or more portions of the flexible computer-assisted system during operation. To determine the pose, or a position and/or orientation, of a portion of the flexible elongate device, the sensor data captured by the sensors may be registered to a frame of reference, such as an anatomical frame of reference used to define locations with respect to anatomical features.

In some cases, it may be desirable to use a flexible manual device rather than a flexible computer-assisted system. For example, a manual flexible device may provide cost advantages relative to computer-assisted systems. In another example, a manual flexible device may be desirable to employ in larger anatomical areas of a patient compared to a flexible computer-assisted system which may be more suited to smaller, peripheral anatomical areas. As a specific example, in the lungs it may be desirable to use a manual bronchoscope in the trachea or bronchi, whereas a flexible computer-assisted system may be operated in the secondary bronchi or smaller portions of the lung. Conventional manual instruments are independent of the flexible computer-assisted system, and are accordingly manually navigated through anatomical structures, and the position of one or more portions of the flexible manual device (e.g., that are inserted within an anatomy) are not known by the operator of the manual device or the computer-assisted system. Even in instances where a flexible manual device includes one or more sensors, the information from the sensors is not registered to a known fiducial marker or reference frame (e.g., a computer-assisted system reference frame, anatomical reference frame, etc.). Accordingly, there is a need to be able to use sensing with flexible manual devices to determine a position and/or movement of the flexible manual instrument relative to a known reference frame to facilitate the use of manual instruments with computer assisted systems.

Techniques described herein may address the problem discussed above. In various instances, the techniques described in this disclosure may provide one or more benefits, alone or in combination with each other. In some embodiments, a flexible elongate device, which may be manually operated, may be registered to a reference frame (e.g., of a computer-assisted system, adapter, portion of the patient, etc.) using information provided by a shape sensor disposed in the flexible elongate device. With the manual elongate device registered to a known reference frame, a computer-assisted system may be used to assist in navigation of the manual device using information from one or more sensors of the device, including the shape sensor. For example, shape information provided by the shape sensor of the flexible elongate device may be compared to a known reference shape (e.g., a shape of an anatomical structure such an internal cavity, external guide adapter, etc.), where the reference shape corresponds to a known location in a desired reference frame, to register the manual elongate device to the desired reference frame.

It should be noted that while techniques described herein refer to manual devices, in some embodiments the techniques described herein may be applied to computer-assisted systems or any flexible elongate device. For example, techniques described herein may be employed to register a second computer-assisted system to the reference frame of a first computer-assisted system. Additionally, manual devices may include one or more actuators (e.g., motors) and corresponding controllers that assist an operator in performing certain functions. In this regard, a “manual device” may refer to a device under direct control of an operator.

In the following detailed description of the aspects of the invention, numerous specific details are set forth in order to provide a thorough understanding of the disclosed embodiments. However, it will be obvious to one skilled in the art that the embodiments of this disclosure may be practiced without these specific details. In other instances, well known methods, procedures, components, and circuits have not been described in detail so as not to unnecessarily obscure aspects of the embodiments of the invention. Therefore, to avoid needless descriptive repetition, one or more components or actions described in accordance with one illustrative embodiment can be used or omitted as applicable from other illustrative embodiments.

The embodiments below will describe various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object. Further, as used herein, the term “distal” means a location closer to a surgical site and the term “proximal” means a location farther away from the surgical site, unless otherwise indicated.

According to exemplary embodiments described herein, position and/or orientation may be measured and discussed with respect to a reference frame. In some cases, a reference frame may be an absolute global reference frame which does not change. For example, a local gravitational direction may establish a global reference frame relative to earth. In some cases, a reference frame may be a local reference frame tied to an orientation or position of a component of a computer-assisted system. For example, a local reference frame may be established based on a table on which a patient lies, or on the orientation of a base of a manipulator arm or cart. In some cases, a reference frame may be a local reference frame tied to an anatomical structure of a patient. For example, a local reference frame may be established based on the pose of an anatomical structure which the computer-assisted system may be operating on. In some cases, determinations may be made with respect to a sensor reference frame (e.g., a shape sensor reference frame). Such a reference frame may be an internal reference frame for a flexible elongate device. In some cases, various reference frames may be registered to one another such that a pose in one reference frame may be understood in the context of another reference frame. Techniques and methods described herein may employ a global reference frame, local reference frame, or a combination thereof, as the present disclosure is not so limited.

While some embodiments are provided herein with respect to such procedures, any reference to medical or surgical instruments and medical or surgical methods is optional and intended as non-limiting. The systems, instruments, and methods described herein may be used for animals, human cadavers, animal cadavers, portions of human or animal anatomy, non-surgical diagnosis, as well as for industrial systems and general robotic, general teleoperational, or robotic medical systems. For example, the systems and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

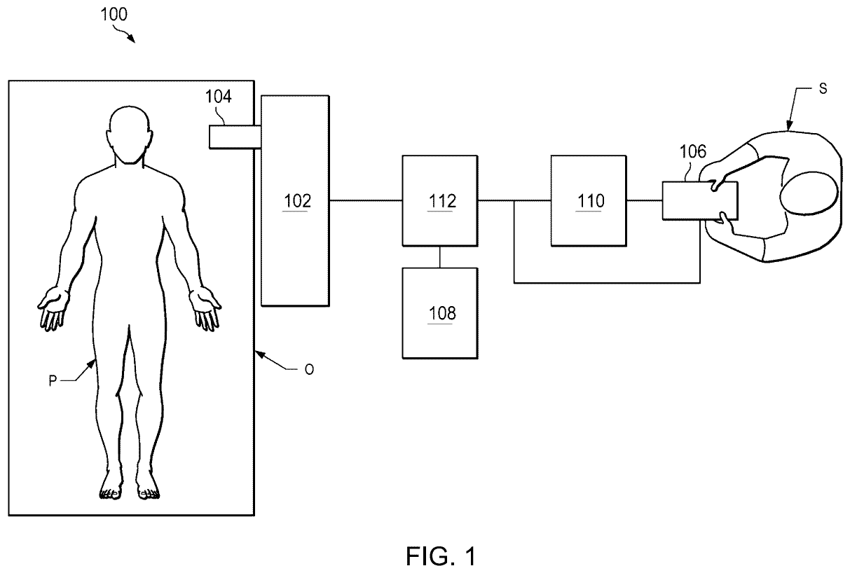

Referring to FIG. 1 of the drawings, a computer-assisted system for use in, for example, surgical, diagnostic, therapeutic, or biopsy procedures, is generally indicated by the reference numeral 100. As shown in FIG. 1, the computer-assisted system 100 generally includes a manipulator assembly 102 for operating a flexible elongate device 104 in performing various procedures on the patient P. The assembly 102 is mounted to or near an operating table O. An operator input system 106 allows the clinician or surgeon S to view the interventional site and to control the manipulator assembly 102.

The operator input system 106 may be located at a surgeon's console which is usually located in the same room as operating table O. However, it should be understood that the surgeon S can be located in a different room or a completely different building from the patient P. Operator input system 106 generally includes one or more control devices for controlling the manipulator assemblies 102. The control devices may include any number of a variety of input devices, such as joysticks, trackballs, data gloves, trigger-guns, hand-operated controllers, voice recognition devices, body motion or presence sensors, or the like. In some embodiments, the control devices will be provided with the same degrees of freedom as the associated flexible elongate devices 104 to provide the surgeon with telepresence, or the perception that the control devices are integral with the flexible elongate devices 104 so that the surgeon has a strong sense of directly controlling devices 104. In other embodiments, the control devices may have more or fewer degrees of freedom than the associated flexible elongate devices 104 and still provide the surgeon with telepresence. In some embodiments, the control devices are manual input devices which move with six degrees of freedom, and which may also include an actuatable handle for actuating instruments (for example, for closing grasping jaws, applying an electrical potential to an electrode, delivering a medicinal treatment, or the like).

The manipulator assembly 102 supports the flexible elongate device 104 and may include a kinematic structure of one or more non-servo-controlled links (e.g., one or more links that may be manually positioned and locked in place, generally referred to as a set-up structure) and a teleoperational manipulator. The manipulator assembly 102 includes plurality of actuators or motors that drive inputs on the flexible elongate device 104 in response to commands from the controller (e.g., a controller 112 which may include one or more processors and associated non-transitory processor readable memory) to control motion of the flexible elongate device 104 in one or more degrees of freedom (e.g., translational, rotational, and/or linear motion). Additionally, the motors can be used to actuate an articulable end effector of the instrument for performing one or more desired operations.

The computer-assisted system 100 also includes a sensor system 108 with one or more sub-systems for receiving information about the instruments of the manipulator assembly. Such sub-systems may include a position/location sensor system (e.g., an electromagnetic (EM) sensor system); a shape sensor system for determining the position, orientation, speed, velocity, pose, and/or shape of the catheter tip and/or of one or more segments along a flexible body of flexible elongate device 104; and/or a visualization system for capturing images from the distal end of the catheter system.

The computer-assisted system 100 may also include a display 110 for displaying an image or representation of the surgical site and flexible elongate device(s) 104 generated by sub-systems of the sensor system 108. In some embodiments, the display 110 may display a virtual navigational image in which the actual location of the flexible elongate device 104 is registered (i.e., dynamically referenced) with the preoperative or concurrent images/model to present the clinician or surgeon S with a virtual image of the internal surgical site from the viewpoint of the location of the tip of the flexible elongate device 104. An image of the tip of the flexible elongate device 104 or other graphical or alphanumeric indicators may be superimposed on the virtual image to assist the surgeon controlling the medical instrument. Alternatively, the flexible elongate device 104 may not be visible in the virtual image.

In other embodiments, the display 110 may display a virtual navigational image in which the actual location of the medical instrument is registered with preoperative or concurrent images to present the clinician or surgeon S with a virtual image of the medical instrument within the surgical site from an external viewpoint. An image of a portion of the medical instrument or other graphical or alphanumeric indicators may be superimposed on the virtual image to assist the surgeon controlling the flexible elongate device 104. As described herein, visual representations of data points may be rendered to the display 110. For example, measured data points, moved data points, registered data points, and other data points described herein may be displayed on the display 110 in a visual representation. The data points may be visually represented in a user interface by a plurality of points or dots on the display or as a rendered model, such as a mesh or wire model created based on the set of data points. In some embodiments, a visual representation may be refreshed in the display 110 after each processing operations has been implemented to alter the data points.

The computer-assisted system 100 also includes a controller 112. The controller 112 includes at least one memory and at least one computer processor (not shown), and typically a plurality of processors, for effecting control between the flexible elongate device 104, the operator input system 106, the sensor system 108, and the display 110. The controller 112 also includes programmed instructions (e.g., a non-transitory computer-readable storage medium storing the instructions) to implement some or all of the methods described in accordance with aspects disclosed herein. While controller 112 is shown as a single block in the simplified schematic of FIG. 1, the system may include two or more data processing circuits with one portion of the processing optionally being performed on or adjacent the manipulator assembly 102, another portion of the processing being performed at the operator input system 106, and the like.

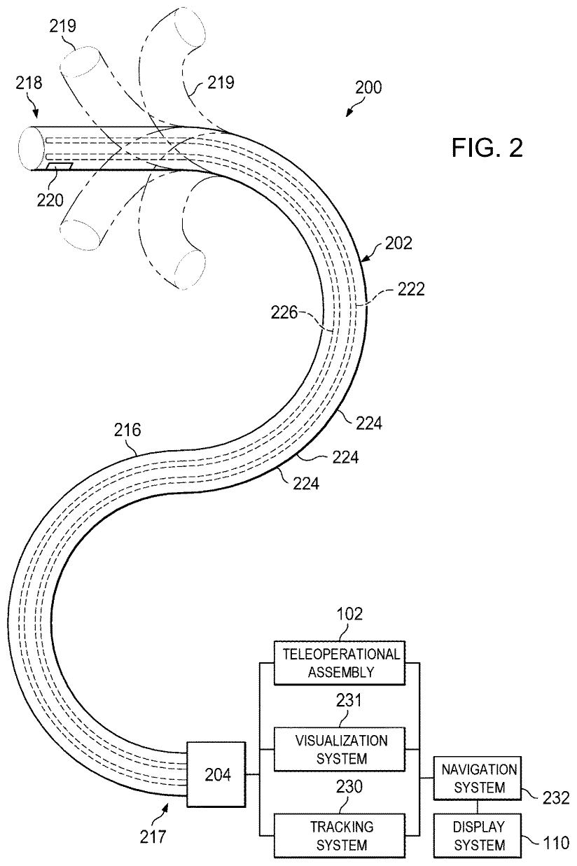

FIG. 2 illustrates a flexible elongate device 200, which may be used as the flexible elongate device 104 in an image-guided medical procedure performed with computer-assisted system 100 shown in FIG. 1 in some embodiments. Alternatively, the flexible elongate device 200 may be used for non-teleoperational exploratory procedures or in procedures involving traditional manually operated medical instruments, such as endoscopy. Additionally or alternatively the flexible elongate device 200 may be used to gather (i.e., measure) a set of data points corresponding to locations with patient anatomic passageways.

The flexible elongate device 200 includes a catheter system 202, or other flexible elongate body, coupled to a housing 204. The catheter system 202 includes an elongated flexible catheter body 216 having a proximal end 217 and a distal end 218 or tip portion. In one embodiment, the flexible body 216 has an approximately 3 mm outer diameter. Other flexible body outer diameters may be larger or smaller. The catheter system 202 may optionally include a shape sensor 222 for determining the position, orientation, speed, velocity, pose, and/or shape of the catheter tip at distal end 218 and/or of one or more segments 224 along the body 216. The entire length of the body 216, between the distal end 218 and the proximal end 217, may be effectively divided into the segments 224. If the flexible elongate device 200 is a flexible elongate device 104 of a computer-assisted system 100, the shape sensor 222 may be a component of the sensor system 108. If the flexible elongate device 200 is manually operated or otherwise used for non-teleoperational procedures, the shape sensor 222 may be coupled to a tracking system 230 that interrogates the shape sensor and processes the received shape data.

The shape sensor 222 may include an optical fiber aligned with the flexible catheter body 216 (e.g., provided within an interior channel (not shown) or mounted externally). In one embodiment, the optical fiber has a diameter of approximately 200 μm. In other embodiments, the dimensions may be larger or smaller. The optical fiber of the shape sensor 222 forms a fiber optic bend sensor for determining the shape of the catheter system 202. In one alternative, optical fibers including Fiber Bragg Gratings (FBGs) are used to provide strain measurements in structures in one or more dimensions. Sensors in alternative embodiments may employ other suitable strain sensing techniques, such as Rayleigh scattering, Raman scattering, Brillouin scattering, and Fluorescence scattering. In other alternative embodiments, the shape of the catheter may be determined using other techniques. For example, the history of the catheter's distal tip pose can be used to reconstruct the shape of the device over the interval of time. As another example, historical pose, position, or orientation data may be stored for a known point of a flexible elongate device along a cycle of alternating motion, such as breathing. This stored data may be used to develop shape information about the catheter. Alternatively, a series of positional sensors, such as electromagnetic (EM) sensors, positioned along the catheter can be used for shape sensing. Alternatively, a history of data from a positional sensor, such as an EM sensor, on the flexible elongate device during a procedure may be used to represent the shape of the instrument, particularly if an anatomic passageway is generally static. Alternatively, a wireless device with position or orientation controlled by an external magnetic field may be used for shape sensing. The history of the wireless device's position may be used to determine a shape for the navigated passageways.

A tracking system 230 may include a position sensor system 220 and a shape sensor 222 for determining the position, orientation, speed, pose, and/or shape of the distal end 218 and of one or more segments 224 along a length of the flexible elongate device 200. The tracking system 230 may be implemented as hardware, firmware, software or a combination thereof which interact with or are otherwise executed by one or more computer processors, which may include the processors of a controller 116.

The flexible catheter body 216 includes a channel 221 sized and shaped to receive a medical instrument 226. In various embodiments, the medical instrument 226 may be an image capture probe that includes a distal portion with a stereoscopic or monoscopic camera at or near the distal end 218 of the flexible catheter body 216 for capturing images (including video images) that are processed by a visualization system 231 for display.

The medical instrument 226 may house cables, linkages, or other actuation controls (not shown) that extend between the proximal and distal ends of the instrument to controllably bend the distal end of the instrument.

The flexible catheter body 216 may also houses cables, linkages, or other steering controls (not shown) that extend between the housing 204 and the distal end 218 to controllably bend the distal end 218 as shown, for example, by the broken dashed line depictions 219 of the distal end. In embodiments in which the flexible elongate device 200 is actuated by a manipulator assembly, the housing 204 may include drive inputs that removably couple to and receive power from motorized drive elements of the manipulator assembly. In embodiments in which the flexible elongate device 200 is manually operated, the housing 204 may include gripping features, manual actuators, or other components for manually controlling the motion of the flexible elongate device 200. The catheter system may be steerable or, alternatively, the system may be non-steerable with no integrated mechanism for operator control of the instrument bending. Also or alternatively, one or more lumens, through which medical instruments can be deployed and used at a target surgical location, are defined in the walls of the flexible body 216. In some embodiments, the catheter body 216 may include one or more articulable portions which are steerable by an operator. In some such embodiments, the catheter body 216 may include flexible non-articulable portions that are not directly steerable by an operator.

In various embodiments, the flexible elongate device 200 may include a flexible bronchial instrument, such as a bronchoscope or bronchial catheter, for use in examination, diagnosis, biopsy, or treatment of a lung. The device 200 is also suited for navigation and treatment of other tissues, via natural or surgically created connected passageways, in any of a variety of anatomic systems, including the colon, the intestines, the kidneys and kidney calices, the brain, the heart, the circulatory system including vasculature, and the like. Accordingly, a flexible elongate device 200 may include catheters, endoscopes, laparoscopes, bronchoscopes, or any other flexible device.

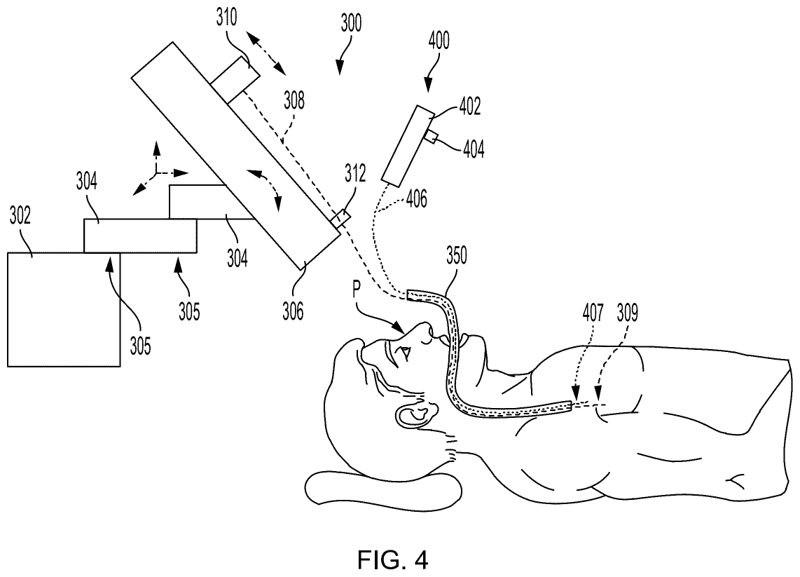

The information from the tracking system 230 may be sent to a navigation system 232 where it is combined with information from the visualization system 231 and/or the preoperatively obtained models to provide the surgeon or other operator with real-time position information on the display 110 for use in the control of the device 200. The controller 116 may utilize the position information as feedback for positioning the device 200. In cases where the flexible elongate device 200 is a manual device, information from the tracking system 230 may be provided to the navigation system 232 after a reference frame of the flexible elongate device 200 is established, as discussed further below with reference to FIGS. 4-9.

In the embodiment of FIG. 2, the device 200 may be teleoperated within the computer-assisted system 100 of FIG. 1. In an alternative embodiment, the manipulator assembly 102 may be replaced by direct operator control. In the direct operation alternative, various handles and operator interfaces may be included for hand-held operation of the instrument.

In alternative embodiments, the computer-assisted system may include more than one manipulator assembly and/or more than one operator input device. The exact number of manipulator assemblies will depend on the medical procedure and the space constraints within the operating room, among other factors. The operator input devices may be co-located, or they may be positioned in separate locations. Multiple operator input devices allow more than one operator to control one or more manipulator assemblies in various combinations.

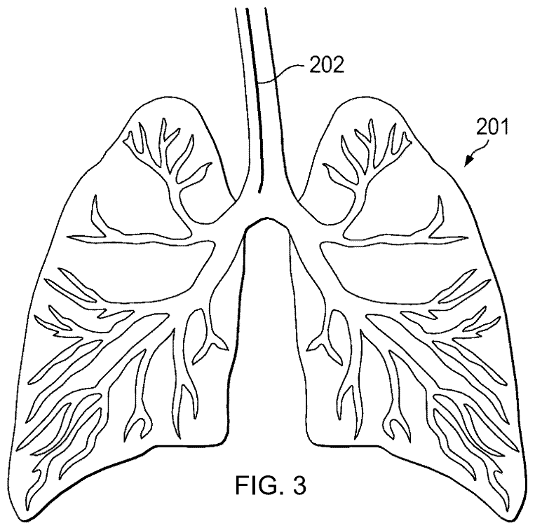

FIG. 3 illustrates the catheter system 202 positioned within an anatomic passageway of a patient anatomy. In this embodiment, the anatomic passageway is an airway of human lungs 201. In alternative embodiments, the catheter system 202 may be used in other passageways of an anatomy.

FIG. 4 depicts a flexible computer-assisted system 300 and a separate flexible elongate device 400, which may be manual, in accordance with embodiments of the present disclosure. As shown in FIG. 4, the flexible computer-assisted system 300 includes a base 302. The base 302 may establish a reference frame for the flexible computer assisted system. That is, the system 300 may coordinate movements, and make determinations of position and orientation (e.g., pose) of various components of the system relative to the base 302. The base 302 may be mounted to a cart, table, or other structure in an operating environment. In some embodiments, the base may be positioned on the ground. The computer-assisted system includes a plurality of links 304. The plurality of links 304 includes a corresponding plurality of joints 305 disposed therebetween that allow the links 304 to move relative to one another in one or more degrees of freedom. In some embodiments, each joint 305 may provide at least one degree of freedom (e.g., a rotational degree of freedom). In some embodiments, the joints 305 may each include an actuator configured to drive the links 304 associated with the joint 305 relative to one another. Accordingly, the system 300 may include a plurality of actuators configured to drive the various links 304 of the system in a plurality of degrees of freedom. The actuators may include any combination of motors (e.g., DC motors, stepper motors, brushless motors, etc.), pneumatic actuators, or other suitable actuators. In some embodiments, the links 304 and joints 305 may provide six degrees of freedom, including the three Cartesian directions and three rotational degrees of freedom about those Cartesian directions (e.g., pitch, roll, and yaw). In other embodiments a flexible computer-assisted system may provide any number of degrees of freedom, greater or less than six.

The computer-assisted system 300 includes a flexible elongate device mount 306. In the specific embodiment of FIG. 4, the mount 306 may be configured to rotate in at least one degree of freedom relative to a distal most link 304. The mount 306 is configured to support a flexible elongate device, not depicted, including a flexible body which may be driven by one or more actuators in the mount and/or by one or more internal actuators. For example, a catheter may be supported by the mount 306. The flexible elongate device may be supported by the mount 306 at an interface 310, which may include a coupling configured to mount the flexible elongate device (e.g., catheter). In some embodiments as shown in FIG. 4, the interface may be configured to move along the mount 306 to move the mounted flexible elongate device in a distal or proximal direction. The system 300 includes a guide 312, which may be used to guide the flexible elongate device. According to the embodiment of FIG. 4, a flexible elongate device includes a shape sensor 308, which is representative of the flexible elongate device (not shown). The shape sensor 308 may provide information regarding a shape of at least a portion of the flexible elongate device to a controller (e.g., at least one processor of the controller) or navigational system. The shape sensor 308 may provide information for determining the position, orientation, speed, velocity, pose, and/or shape of one or more portions of the flexible elongate device along its length, and for determining a position and/or pose of a distal end 309 of the flexible elongate device. The flexible elongate device including shape sensor 308 may extend into an anatomical structure of a patient P (e.g., an airway). An endotracheal tube 350, or other structure, may provide a pathway into the patient P. As elaborated on below, the endotracheal tube, or other structure may have a known reference shape that may be measured or otherwise obtained. In some embodiments, the endotracheal tube 350 may also attach to the mount 306.

As shown in FIG. 4, the separate flexible elongate device 400, which again may be a manual flexible elongate device, may include a handle 402 and a manipulator 404 configured to be grasped by an operator. The handle 402 may be coupled to a flexible elongate body, not depicted for clarity, such as a catheter, endoscope laparoscope, etc. The manipulator 404 may be used to steer and/or operate the flexible elongate body and/or any associated instruments. The manipulator 404 may be a knob, button, lever, or other suitable structure configured to allow an operator to use the device 400. In some embodiments, the manipulator 404 may be employed to articulate an articulable portion of the flexible elongate device 400, such as a distal portion of the flexible elongate device 400 along pitch and yaw axes. In some embodiments, multiple manipulators may be employed on a handle 402. As shown in FIG. 4, the flexible elongate device 400 includes a shape sensor 406 which may provide information regarding a shape of at least a portion of the flexible elongate device 400 to a controller (e.g., at least one processor of the controller) or navigational system. In some embodiments, the shape sensor 406 may provide information to a controller and/or navigational system of the computer-assisted system 300. The shape sensor 406 may provide information for determining the position, orientation, speed, velocity, pose, and/or shape of the one or more portions disposed along a length of the separate flexible elongate device 400 relative to an established reference frame, as discussed further below. For example, the shape sensor 406 may be employed to determine the position and/or orientation of a distal end 407 of the flexible elongate device 400 relative to a known reference frame using information related to a shape of one or more portions of the flexible elongate device 400. As shown in FIG. 4, the flexible body of the elongate device including the shape sensor 406 may extend into an anatomical structure of a patient P (e.g., an airway). In the embodiment of FIG. 4, the flexible body and shape sensor 406 extends through an endotracheal tube 350.

According to the embodiment of FIG. 4, the shape sensor 406 of the separate flexible elongate device 400 may be employed to register the position and/or orientation of the flexible body of the flexible elongate device 400 (e.g., a position and/or orientation of the distal end 407). In some embodiments, a controller may receive information from the shape sensor 406 and may use that information to determine a position of a portion of the flexible elongate device 400.

In some embodiments as discussed above, information related to anatomical structures (e.g., lumens such as internal cavities) of a patient P may be employed for the navigation of a computer-assisted system through the patient. That is, information from imaging or scans may be employed to generate a three-dimensional model of the anatomy of a particular patient. The three-dimensional model may include anatomical features and corresponding positions of those features, thus providing an anatomical reference frame for defining positions with respect to the features in the three-dimensional model. In some embodiments, a reference shape of an anatomical or non-anatomical structure may be registered to the anatomical reference frame (e.g., by the controller). For example, a specific geometry of the structure may be registered to the anatomical reference frame (e.g., a pose of the geometry in three-dimensional space may be established relative to the patient P). The reference shape may be defined by a set of points, each point corresponding to a location on a shape sensor. Each point of the reference shape is defined in the anatomical reference. In some embodiments, a reference shape may be determined based on the specific geometry of a reference object, such as a trachea, bronchus, secondary bronchi, an endotracheal tube disposed in a patient, or other lumen present within the anatomy of a patient. In such an embodiment, anatomical structures used for a reference shape may have a series of directional changes, three-dimensional shape, or other set of parameters which is uniquely identifiable within the anatomy. That is, the reference shape may be a shape that corresponds to features of the three-dimensional model and thus is present in a known location in the anatomical reference frame. In other embodiments, the reference shape may be a shape of a flexible elongate structure within the reference object. For example, rather than the shape of the structure itself, the registered shape may be the shape that a flexible body adopts when passing through the anatomical structure, endotracheal tube, or other structure having known position in the anatomical reference frame. In some embodiments, such a shape may be initially obtained by a shape sensor of a computer-assisted system 300 with a known reference frame (e.g., a local reference frame of the computer-assisted system), such as by passing the elongate device including shape sensor 308 through the reference object. For example, the shape sensor 308 may be employed to measure the shape of a lumen of the patient P which may be used as the reference shape. In some embodiments, the reference shape may be obtained from a prior procedure (e.g., using a computer-assisted system 300) where a shape of a reference structure such as a lumen was measured by a shape sensor. In some embodiments, the reference shape may be registered to a reference frame other than an anatomical reference frame, such a computer-assisted system 300 reference frame or some other reference frame.

With a reference shape established, the shape sensor 406 of the manual flexible elongate device 400 may be used to determine a pose of the one or more portions of the flexible elongate device 400 with reference to the registered reference frame. As the flexible elongate device 400 is inserted into the patient P, the shape sensor 406 may conform to and measure the shape of the lumen in which the shape sensor 406 and the associated flexible elongate body, not depicted, of the flexible elongate device 400 is disposed. The shape sensor 406 may provide shape information to a controller regarding its pose in a shape sensor reference frame. For example, each point in the shape information may be defined with respect to the starting point of the shape sensor 406 as the origin of the shape sensor reference frame. As the shape sensor 406 passes through the reference structure, which in the depicted embodiment is endotracheal tube 350, the shape information provided by the shape sensor 406 may be compared to the reference shape. Exemplary comparison techniques include, but are not limited to, point matching (e.g., iterative closest point), vector matching, defining a volume and matching points within the volume, and spline parameter recognition. Based on the comparison, it may be determined if the shape of the shape sensor 406 matches the reference shape. If the reference shape matches the shape of the shape sensor 406, the pose of the portion of the shape sensor 406, and the corresponding portion of the flexible elongate body of the device, matching the reference shape may be registered to the desired reference frame (e.g., an anatomical reference frame). That is, the positions of the reference shape in desired reference frame are known, and thus the points of the shape sensor information (e.g., initially defined in the shape sensor reference frame) for a portion of the shape sensor 406 that conform to the reference shape can be correlated with the desired reference frame. Furthermore, other non-conforming portions of the shape sensor 406 (e.g., more distal portions that do not conform to the reference shape) can be correlated to the desired reference frame based on the shape information defining the spatial relationship between the conforming and non-conforming portions in the shape sensor reference frame. Furthermore, as the flexible elongate device 400 is inserted or retracted, different portions of the flexible elongate device 400 (and shape sensor 406) may conform to the reference shape, thus facilitating the translation between the shape sensor reference frame and the desired reference frame for points along the flexible elongate device 400 (and shape sensor 406).

In some embodiments, an anatomical reference frame may include a reference landmark associated with a reference shape. This reference landmark may be located at a proximal portion of an interval cavity of a patient in some embodiments (e.g., mouth, esophagus, trachea, bronchus, etc.). In some embodiments, a comparison of the shape of a flexible elongate device with a reference shape of the reference landmark may be employed to generate a similarity threshold. In some such embodiments, when the similarity threshold exceeds a threshold value the shape of the shape sensor 406 and the reference shape may be considered matching. The level of the similarity threshold may be based on the particular comparison technique used as well as the geometry and scale of the reference shape. Once the reference shape is identified at a portion of the shape sensor 406, information from the shape sensor 406 may be used to determine position, orientation, speed, velocity, pose, and/or shape of the one or more portions along the separate flexible elongate device relative to the established reference shape. Accordingly, navigation systems (e.g., of the computer-assisted system 300) may be used while operating the manual flexible elongate device 400.

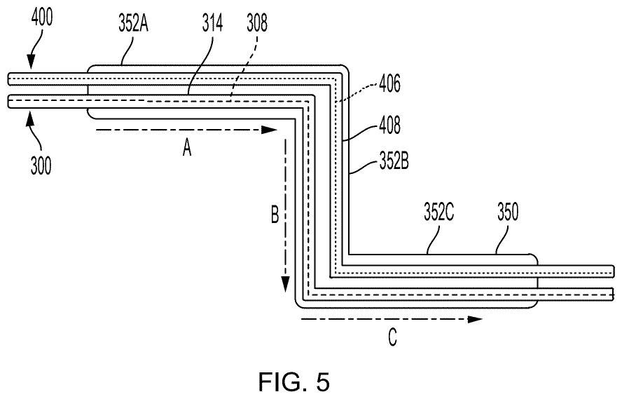

As noted above, the reference object may be an anatomical structure, or some other structure associated with the anatomical features of a patient. In some embodiments, a reference shape may be established based on a structure inserted into the patient. In such embodiments, the reference shape may not be an anatomical structure but rather a structure associated with a patient. For example, in the embodiment of FIG. 4, the endotracheal tube 350 may be employed as the reference shape. In some instances, use of a non-anatomical structure may provide a more consistent and detectable reference shape. For example, the endotracheal tube 350 may have an S-shape which is readily identifiable with information from a shape sensor. In the case of vector matching, the endotracheal tube may be identified with two or more (e.g., three) vectors in series with approximately 90-degree rotations from one another, as will be discussed further with reference to FIG. 5. While an endotracheal tube is described in embodiments herein, any suitable structure may be employed as a reference shape in other embodiments.

In some embodiments as shown in FIG. 4, a shape sensor 308 of a computer-assisted system 300 may be disposed in a patient P concurrently with a shape sensor 406 of the manual flexible elongate device 400. In such a case, a reference shape may be measured by shape sensor 308 and compared to a shape of the shape sensor 406 of the manual flexible elongate device 400. In this manner, any reference frame in which the pose of the shape sensor 308 is known may be used to register the pose of the shape sensor 406 of the manual flexible elongate device 400 to that same reference frame. In other embodiments, the computer assisted system 300 may be used and removed from the patient P prior to the use of the manual flexible elongate device 400. In such embodiments, the reference shape may be measured by the shape sensor 308 and stored (e.g., in non-transitory processor readable memory) for later comparison to the shape information provided by the shape sensor 406 of the separate flexible elongate device 400. In some embodiments, a reference shape may be established without measurement by the shape sensor 308 of the computer-assisted system 300.

In some embodiments, the separate shape sensor 406 of the manual flexible elongate device 400 connects with an interrogator (e.g., a multi-core fiber interrogator) at the controller 112. The interrogator may include an embedded system including electronics, fiber optics and processing nodes used to measure shape of a shape sensor. The mount 306 may be collapsed and the links 304 and joints 305 may be adjusted for placement near the patient P. The base of the shape sensor 406 is fixtured to the mount 306 using a calibration and the known location of the mount 306 in the anatomical reference frame is used register the shape sensor 406 to the anatomical reference frame. This links the manual flexible elongate device 400 to the same kinematic frame as the flexible elongate device that includes the shape sensor 308, and kinematics may be used to maintain the initial registration. In some embodiments, the manual flexible elongate device 400 uses the shape sensor 308 of the flexible computer-assisted system 300 rather than a separate shape sensor 406. For example, the flexible elongate device 400 may include a lumen through which the shape sensor 308 is inserted. Here, the registration of the shape sensor 308 to the anatomical reference frame may be used to register the manual flexible elongate device 400 to the anatomical reference frame.

FIG. 5 depicts a schematic of a flexible computer-assisted system 300 and a separate flexible elongate device 400 disposed in a lumen in accordance with embodiments of the present disclosure. In particular, FIG. 5 depicts portions of a flexible computer-assisted system and a flexible manual device in an endotracheal tube 350. The flexible elongate device 400 is configured to conform to the lumen of the endotracheal tube 350 or other structure. The computer-assisted system includes a flexible body 314 housing a shape sensor 308. The flexible body 314 may include one or more articulable portions allowing the flexible body to be navigated through lumens of a patient. The separate flexible elongate device 400 includes a flexible body 408 housing a shape sensor 406. The flexible body 408 may include one or more articulable portions allowing the flexible body 408 to be steered through lumens of a patient. The shape sensors 308, 406 may provide information regarding the pose of the flexible body 314 and flexible body 408, respectively. As the flexible bodies are disposed in the endotracheal tube 350, the shape sensors 308, 406 provide indirect information regarding the shape of the endotracheal tube. Accordingly, where the endotracheal tube is employed as a reference shape, the shape sensors 308, 406 may provide information regarding a shape of portions of the flexible bodies that match the reference shape.

As shown in FIG. 5, an endotracheal tube 350 may be employed as a reference shape in some embodiments. The endotracheal tube may include a series of three straight portions 352A, 352B, 252C, where each distal portion is angled relative to its immediately proximal portion. For example, a first straight portion 352A of the endotracheal tube may be angled relative to a second straight portion 352B. Likewise, the third straight portion 352C is angled relative to the second straight portion 352B. An endotracheal tube, adopting the geometry of the trachea of a patient, may be represented as a series of vectors as shown in FIG. 5. In particular, the tube 350 extends in a first vector A. Next, the tube 350 extends in a second vector B, which may be approximately perpendicular to the first direction. Lastly, the tube 350 extends in a third vector C, which may be approximately perpendicular to the second vector B, and parallel to the first vector A. According to such an embodiment, a controller may compare shape information provided by the shape sensor 406 of the manual flexible elongate device 400 using vector matching to this series of vectors. For example, where a portion of the shape sensor 406 is aligned with the three vectors A, B, C it may be determined that the portion matches the reference shape. Based on that match, the pose of the other portions of the shape sensor 406 may be determined relative to the identified reference shape. It should be noted that while the structures shown in FIG. 5 are illustrated with sharp corners, other types of shapes including shapes with curved transitions between the different portions, such as might be present in an endotracheal tube, may also be used. Furthermore, an endotracheal tube may include different numbers of portions.

In some embodiments, vector matching techniques may be employed as a part of identifying a reference shape at a portion of a flexible elongate device. In some embodiments, such a technique may include identifying a plurality of reference vectors in the reference shape. For example, the vectors A, B, C may be identified as reference vectors. The technique may also include identifying a plurality of measured vectors at the first portion. The vectors may be identified from shape information provided by the shape sensor 406. The technique may also include comparing the relative directions of the plurality of reference vectors and the plurality of measured vectors. For example, where a portion of the shape sensor 406 extends along vectors that are substantially similar to the reference vectors, a match may be identified.

While in some embodiments vector matching may be employed as a part of identifying a reference shape at a portion of a flexible elongate device, in other embodiments other techniques such as point matching may be employed. In some embodiments, such a technique may include identifying a plurality of reference points in the reference shape. The reference points may form a three-dimensional point cloud, in some embodiments. The technique may also include identifying a plurality of measured points at the first portion. For example, three-dimensional points may be obtained from shape sensor 406. The technique may further include comparing the relative positions of the plurality of reference points and the plurality of measured points. Such a comparison may include suitable algorithms, such as iterative closest point. As discussed previously, when employing such algorithms, a similarity threshold may be used. For example, identifying the reference shape at the first portion may include matching the plurality of measured points to the plurality of reference points within a similarity threshold. The similarity threshold may be established based on the shape sensor employed and the particular algorithm used. The similarity threshold may be a percentage (e.g., between 0 and 100% in some embodiments).

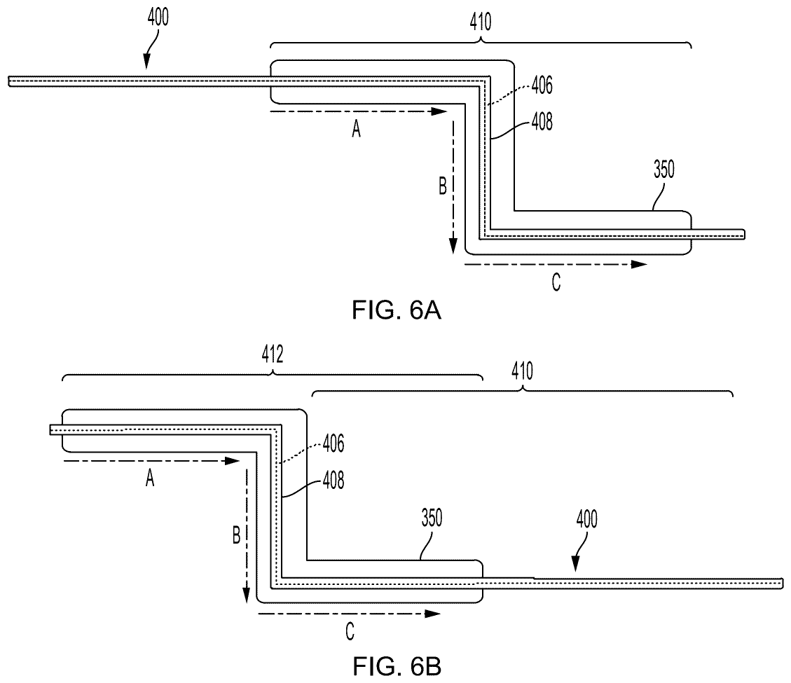

FIG. 6A depicts a schematic of a flexible manual elongate device 400 in a first state disposed in a lumen (e.g., an endotracheal tube 350) and FIG. 6B depicts a schematic of the flexible manual device in a second state. FIGS. 6A-6B depict how the position of the reference shape along a shape sensor 406 changes as the flexible manual elongate device is used. As shown in FIG. 6A, the device 400 includes a flexible body 408 which extends through the endotracheal tube 350, which may function as the reference shape as discussed above with reference to FIG. 5. The flexible body 408 extends proximally (e.g., to the left of the page) and distally (e.g., to the right of the page from the endotracheal tube). Accordingly, only a first portion 410 of the shape sensor 406 is disposed in the endotracheal tube. As shown in FIG. 6B, if the flexible body 408 is advanced distally (e.g., to the right side of the page), the portion of the shape sensor 406 aligned with the reference shape changes. In particular, a second portion 412, proximal the first portion 410, is now aligned with the reference shape. Accordingly, as a flexible manual elongate device is used, the shape of portions of the shape sensor 406 may be continuously or iteratively (e.g., after a predetermined time period) compared to the reference shape to ensure pose information determined from the shape sensor 406 is accurate relative to the desired reference frame. In performing such comparisons, a controller may identify the reference shape and associate it with a particular portion of the shape sensor 406 disposed along a length of the flexible elongate device 400.

During a registration process, a controller may be configured to measure the shape of a flexible elongate device 400 with a shape sensor 406 as noted above. The information provided by the shape sensor 406 may be in a shape sensor reference frame prior to registration or conversion to other reference frames. As discussed previously, the reference shape may be identified as being associated with a portion of the shape sensor 406. Accordingly, the reference shape may be registered to the shape sensor reference frame. As the reference shape position is known in the anatomical reference frame (or another desired reference frame), the shape sensor reference frame may be registered to the anatomical reference frame using the known position and/or pose of the reference shape. With the reference shape position and/or pose established, the controller may determine a position and/or pose of one or more portions (e.g., portion 410 in FIG. 6B) of the separate flexible elongate device 400 disposed distally or proximally from the reference shape in the shape sensor reference frame with the shape sensor 406. The controller may then register the position and/or pose of these one or more other portions of the flexible elongate device 400 with the anatomical reference frame using the known position and/or pose of the reference shape and a spatial relationship between the portion of the flexible elongate device 400 associated with the reference shape and the other portions (e.g., a spatial relationship between the first portion 410 and the second portion 412). Depending on the embodiment, the spatial relationship may correspond to a sensed spatial relationship measured with the shape sensor 406. In some embodiments, the second portion may be a distal end of the flexible elongate device 400.

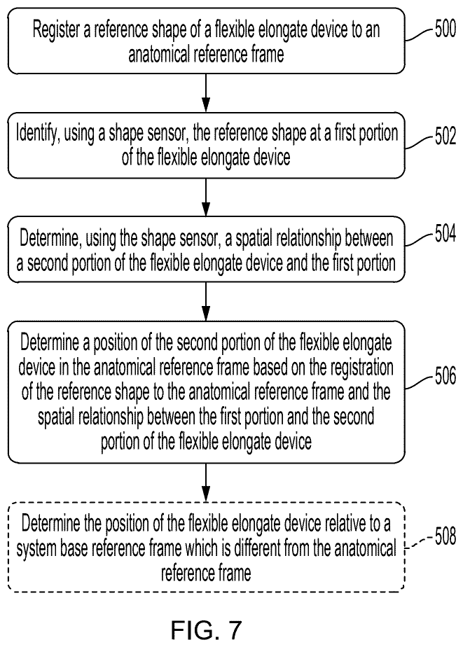

FIG. 7 is a flow chart for an embodiment of tracking a medical system comprising a flexible elongate device. In block 500, the method includes registering a reference shape of a flexible elongate device to an anatomical reference frame. The flexible elongate device may be a manual flexible elongate device in some embodiments. The reference shape may be obtained using the flexible elongate device or based on previously obtained information associated with another flexible elongate device (e.g., that has a shape sensor which has been registered to the anatomical reference frame), such as a computer-assisted elongate device. In block 502, the method includes identifying the reference shape at a first portion of the flexible elongate device. The identification may be made using information from a shape sensor disposed in the flexible elongate device. As discussed previously, the identification may be made using one or more techniques, including point matching and/or vector matching. In block 504, the method includes determining a spatial relationship between a second portion of the flexible elongate device and the first portion of the elongate device. For example, a shape sensor reference frame may be employed to determine a position and/or orientation (e.g., a pose) of the second portion relative to a position and/or orientation of the first portion. In some embodiments, the shape sensor may provide information regarding the spatial relationship between the first portion and the second portion of the flexible elongate device. In block 506, the method includes determining a position of the second portion of the flexible elongate device in the anatomical reference frame based on the registration of the reference shape to the anatomical reference frame and the spatial relationship between the first portion and the second portion of the flexible elongate device. In optional block 508, the method includes determining the position of the flexible elongate device relative to a system base reference frame which may be different from the anatomical reference frame. For example, a computer-assisted system may have a system base reference frame that is established separate from the anatomical reference frame. In some embodiments, the computer-assisted system may also include a shape sensor that may be used to identify the reference shape within the system base reference frame. Accordingly, the reference shape when combined with information from the shape sensor of a flexible elongate device may be used to determine position and/or orientation information of the flexible elongate device in a shape sensor reference frame, an anatomical reference frame, and/or a system base reference frame.

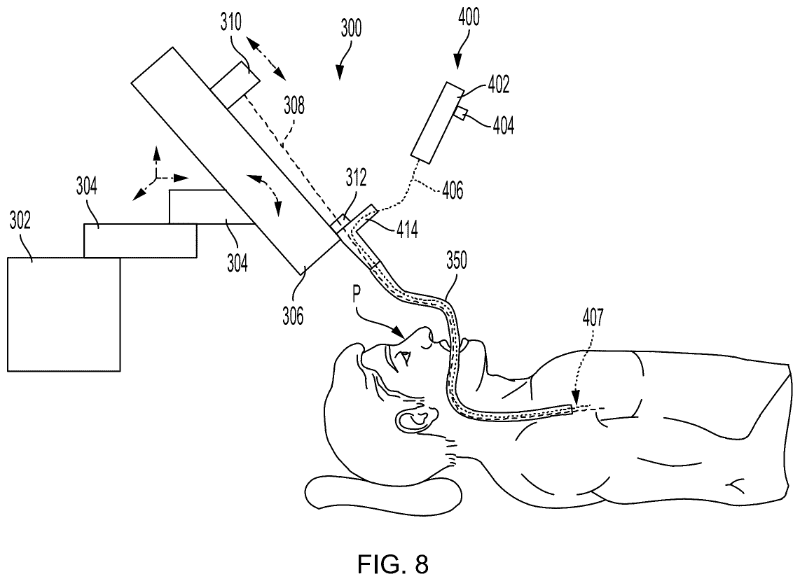

FIG. 8 depicts a flexible computer-assisted system 300 and a flexible manual device 400 in accordance with embodiments of the present disclosure. As shown in FIG. 8, the flexible computer-assisted system 300 is similar to that described with reference to FIG. 4. The system 300 includes a base 302. The base 302 may establish a reference frame for the flexible computer assisted system. That is, the system 300 may coordinate movements, and make determinations of position and orientation (e.g., pose) of various components of the system relative to the base 302. The base 302 may be mounted to a cart, table, or other structure in an operating environment. In some embodiments, the base may be positioned on the ground. As shown in FIG. 8, the computer-assisted system also includes a plurality of links 304 and joint 305. The plurality of links includes a corresponding plurality of joints 305 that allow the links to move relative to one another in one or more degrees of freedom. In some embodiments, the links 304 and joints 305 may provide six degrees of freedom, including the three Cartesian directions and three rotational degrees of freedom about those Cartesian directions (e.g., pitch, roll, and yaw). In other embodiments a flexible computer-assisted system may provide any number of degrees of freedom, greater or less than six.

As shown in FIG. 8, the computer-assisted system 300 includes a flexible elongate device mount 306 as discussed in FIG. 4. A flexible elongate device, not depicted for clarity, may be coupled to the mount 306 at an interface 310, which may include a coupling configured to mount a catheter or other flexible elongate device. As shown in FIG. 8, the system 300 includes a guide 312, which may be used to guide the flexible elongate device of the computer-assisted system. According to the embodiment of FIG. 8, the flexible elongate device of the computer-assisted system includes a shape sensor 308, which is representative of the flexible elongate device (not shown). The shape sensor 308 may provide information regarding a shape of at least a portion of the flexible elongate device to a controller (e.g., at least one processor of the controller) or navigational system. The shape sensor 308 may provide information for determining the position, orientation, speed, velocity, pose, and/or shape of the one or more portions along the flexible elongate device, and for determining a position of a distal end 309 of the flexible elongate device. As shown ion FIG. 8, the flexible elongate device including shape sensor 308 may extend into an anatomical structure of a patient P (e.g., an airway). As shown in FIG. 8, the endotracheal tube 350 may provide a pathway into the patient P.

According to the embodiment of FIG. 8, the endotracheal tube 350 may be attached to the computer-assisted system 300. That is, a proximal portion of the endotracheal tube is at a known pose relative to the system base reference frame. Accordingly, such an arrangement may be employed with techniques described above to register a shape sensor reference frame of the separate flexible elongate device 400 to a system base reference frame, without necessarily registering the shape sensor reference frame to an anatomical reference frame.

As shown in FIG. 8, the separate flexible elongate device 400 includes a handle 402 and a manipulator 404 configured to be grasped by an operator, as discussed with reference to FIG. 4. The handle 402 may be coupled to a flexible elongate body, not depicted for clarity, such as a catheter, endoscope laparoscope, etc. The manipulator 404 may be used to steer and/or operate the flexible body 408 and/or any associated instruments. As shown in FIG. 8, the device 400 includes a shape sensor 406 which may provide information regarding a shape of at least a portion of the separate flexible elongate device 400 to a controller (e.g., at least one processor of the controller) or navigational system. In some embodiments, the shape sensor may provide information to a controller and/or navigational system of the computer-assisted system 300. The shape sensor 406 may provide information for determining the position, orientation, speed, velocity, pose, and/or shape of the one or more portions along the separate flexible elongate device 400 relative to a sensor reference frame or other registered reference frame, as discussed herein. For example, the shape sensor 406 may be employed to determine the position and/or orientation of a distal end 407 of the flexible elongate device 400 relative to a known reference frame. The flexible elongate body of the flexible elongate device 400 including the shape sensor 406 may extend into an anatomical structure of a patient P (e.g., an airway). In the embodiment of FIG. 8, the flexible elongate body and shape sensor 406 extends through an endotracheal tube 350.

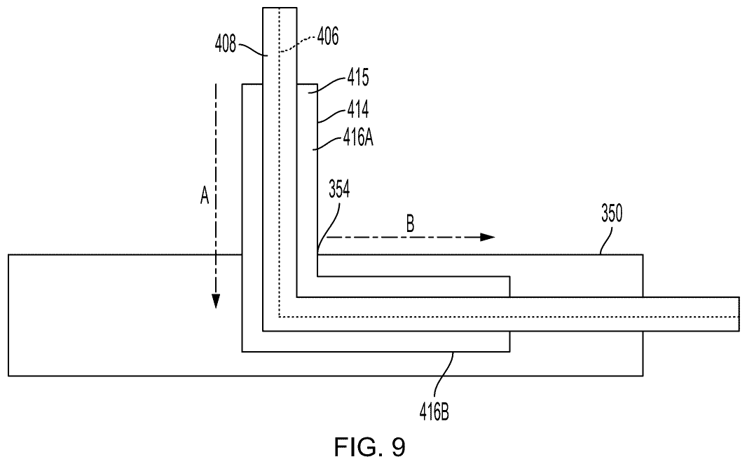

According to the embodiment of FIG. 8, an adapter 414 may be disposed between and connect the endotracheal tube 350 to the mount 306. The adapter 414 may include a primary lumen (e.g., straight portion 416B of FIG. 9) passing therethrough that is configured to permit a flexible elongate device (not depicted) connected to the mount 306 to pass through the primary lumen through the adapter 414 and into the endotracheal tube or other appropriate structure. The adapter 414 may also include a secondary lumen (e.g., straight portion 416A of FIG. 9) connected with the primary lumen that is configured to permit a separate flexible elongate device 400, which may be manually operated in some embodiments, to be inserted into the endotracheal tube or other structure through the adapter 414 as well. The adapter 414 may be configured to function as a known shape and pose within a desired reference frame. For example, as shown in FIG. 8, the adapter 414 may be disposed on a portion of the computer-assisted system 300, such that the pose of the adapter 414 is known with respect to a system base reference frame. In other embodiments, the adapter 414 may be positioned at a known pose relative to an anatomical reference frame. For example, the adapter 414 may be placed in the mouth of a patient or positioned and oriented at a particular pose with respect to the patient. Accordingly, when the shape of a lumen of the adapter 414 is identified by a flexible elongate device positioned therein using techniques described herein, a shape sensor reference frame may be registered to the desired reference frame (e.g., anatomical reference frame or system base reference frame). In some embodiments, the adapter 414 may be sufficiently rigid, such that the shape of the adapter 414 does not significantly change when oriented in different orientations or if forces are applied to the adapter 414 (i.e., the shape may still be identified using the disclosed techniques). In this manner, the shape of the adapter 414 may be known, as the adapter 414 may be designed to have a particular identifiable shape. In the embodiment of FIG. 8 and as will be discussed further with reference to FIG. 9, the adapter 414 may include first and second straight portions angled relative to one another. Such an arrangement may provide a readily identifiable reference shape for use with shape sensors as described herein, though other identifiable shapes may also be used.

FIG. 9 depicts a schematic of a flexible manual device 400 disposed in an adapter 414 in accordance with embodiments of the present disclosure. The adapter 414 includes a lumen 415. A flexible body 408 of the flexible elongate device 400 is disposed in and extends through the lumen 415. The flexible elongate device 400 also includes a shape sensor 406 configured to provide orientation and/or position information along a length of the flexible elongate body 408 of the flexible elongate device 400 within a shape sensor reference frame. As shown in FIG. 9, the adapter 414 may include a first straight portion 416A and a second straight portion 416B. the second straight portion is angled relative to the second straight portion. In the embodiment of FIG. 9, the second straight portion 416B is angled at 90-degrees relative to the first straight portion 416A. In other embodiments, any suitable angle may be employed, including acute or oblique angles. As shown in FIG. 9, the flexible elongate body 408 of the flexible elongate device 400 conforms to the shape of the lumen 415 of the adapter 414. As shown in FIG. 9, the first straight portion 416A extends along a first vector A. The second straight portion 416B extends along a second vector B. The first and second vectors may be employed for reference shape matching as discussed previously. That is, information from the shape sensor 406 may be employed to identify a reference shape along a portion of the shape sensor based on matching the first vector A and the second vector B. In other embodiments any number of vectors may be employed (e.g., three as discussed above with reference to FIG. 5). Of course, while a particular shape is shown in the figure, any shape for a lumen 415 passing through an adapter 414 or other structure may be used with the various embodiments disclosed herein.

In the embodiment of FIG. 9, the adapter 414 is disposed in a port 354 formed in an endotracheal tube 350. In other embodiments, the adapter 414 may be disposed in another structure. In some embodiments, the adapter 414 may be mounted to a portion of a computer-assisted system. The adapter 414 may provide a known pose within a desired reference frame. For example, if the adapter 414 is disposed on a portion of a computer-assisted system, the pose of the adapter 414 is known with respect to a system base reference frame. In other embodiments, the adapter 414 may be positioned at a known pose relative to an anatomical reference frame. For example, the adapter 414 may be placed in the mouth of a patient or positioned and oriented at a particular pose with respect to the patient. In the embodiment of FIG. 9, the endotracheal tube 350 may be positioned at a particular pose, which may provide a known pose for the adapter 414 disposed in the endotracheal tube. Accordingly, when the shape of the adapter 414 (e.g., the reference shape) is identified using techniques described herein with a shape sensor, a shape sensor reference frame may be registered to the desired reference frame (e.g., anatomical reference frame or system base reference frame).

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

The terms “program” or “software” are used herein in a generic sense to refer to any type of computer code or set of computer-executable instructions that can be employed to program a computer or other processor to implement various aspects of the present disclosure as discussed above. Additionally, it should be appreciated that according to one aspect of this embodiment, one or more computer programs that when executed perform methods of the present disclosure need not reside on a single computer or processor but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “operator.” It should be appreciated that a “operator” need not be a single individual, and that in some embodiments, actions attributable to a “operator” may be performed by a team of individuals and/or an individual in combination with computer-assisted tools or other mechanisms.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A medical tracking system comprising:
a flexible elongate device;
a shape sensor configured to measure a shape of the flexible elongate device; and
a controller comprising at least one processor, the controller configured to:
register a reference shape of the flexible elongate device to an anatomical reference frame;
identify, using the shape sensor, the reference shape at a first portion of the flexible elongate device;
determine, using the shape sensor, a spatial relationship between a second portion of the flexible elongate device and the first portion; and
determine a position of the second portion of the flexible elongate device in the anatomical reference frame based on the registration of the reference shape to the anatomical reference frame and the spatial relationship between the first portion and the second portion of the flexible elongate device.

2. The medical tracking system of claim 1, wherein the flexible elongate device includes an articulable portion configured to be manually articulated by an operator.

3. The medical tracking system of claim 1, wherein registering the reference shape comprises measuring the shape of a second elongate device with a second shape sensor.

4. The medical tracking system of claim 1, wherein the reference shape is a lumen shape of a lumen of a reference object, wherein the flexible elongate device is configured to conform to the lumen shape when disposed in the lumen.

5. The medical tracking system of claim 4, wherein the reference object is an adapter having a known shape and pose in the anatomical reference frame.

6. The medical tracking system of claim 5, wherein the adapter comprises a first straight portion and a second straight portion angled relative to the first straight portion.

7. The medical tracking system of claim 4, wherein the reference object is an endotracheal tube.

8. The medical tracking system of claim 1, wherein identifying the reference shape at the first portion comprises:
identifying a plurality of reference points in the reference shape;
identifying a plurality of measured points at the first portion; and
comparing the relative positions of the plurality of reference points and the plurality of measured points.

9. The medical tracking system of claim 8, wherein identifying the reference shape at the first portion comprises matching the plurality of measured points to the plurality of reference points within a similarity threshold.

10. The medical tracking system of claim 1, wherein identifying the reference shape at the first portion comprises:
identifying a plurality of reference vectors in the reference shape;
identifying a plurality of measured vectors at the first portion; and
comparing the relative directions of the plurality of reference vectors and the plurality of measured vectors.

11. The medical tracking system of claim 1, wherein the anatomical reference frame includes a reference landmark located at a proximal portion of an internal cavity of a patient.

12. The medical tracking system of claim 1, wherein the controller is further configured to determine the position of the flexible elongate device relative to a system base reference frame different from the anatomical reference frame.

13. The medical tracking system of claim 1, wherein the second portion is disposed distal relative to the first portion.

14. A non-transitory computer-readable storage medium storing instructions that, when executed by at least one processor associated with a computer-assisted device, causes the at least one processor to perform a method of tracking a medical system comprising a flexible elongate device, the method comprising:
registering a reference shape of the flexible elongate device to an anatomical reference frame;
identifying, using a shape sensor, the reference shape at a first portion of the flexible elongate device;
determining, using the shape sensor, a spatial relationship between a second portion of the flexible elongate device and the first portion; and
determining a position of the second portion of the flexible elongate device in the anatomical reference frame based the registration of the reference shape to the anatomical reference frame and the spatial relationship between the first portion and the second portion of the flexible elongate device.

15. (canceled)

16. The non-transitory computer-readable storage medium-method of claim 14, wherein registering the reference shape comprises measuring the shape of a second elongate device with a second shape sensor.

17. (canceled)

18. (canceled)

19. (canceled)

20. (canceled)

21. The non-transitory computer-readable storage medium of claim 14, wherein identifying the reference shape at the first portion comprises:
identifying a plurality of reference points in the reference shape;
identifying a plurality of measured points at the first portion; and
comparing the relative positions of the plurality of reference points and the plurality of measured points.

22. The non-transitory computer-readable storage medium of claim 21, wherein identifying the reference shape at the first portion comprises matching the plurality of measured points to the plurality of reference points within a similarity threshold.

23. The non-transitory computer-readable storage medium of claim 14, wherein identifying the reference shape at the first portion comprises:
identifying a plurality of reference vectors in the reference shape;
identifying a plurality of measured vectors at the first portion; and
comparing the relative directions of the plurality of reference vectors and the plurality of measured vectors.

24. (canceled)

25. The non-transitory computer-readable storage medium of claim 14, further comprising determining the position of the flexible elongate device relative to a system base reference frame different from the anatomical reference frame.

26. (canceled)

27. A method of tracking a medical system comprising a flexible elongate device, the method comprising:
registering a reference shape of the flexible elongate device to an anatomical reference frame;
identifying, using a shape sensor, the reference shape at a first portion of the flexible elongate device;
determining, using the shape sensor, a spatial relationship between a second portion of the flexible elongate device and the first portion; and
determining a position of the second portion of the flexible elongate device in the anatomical reference frame based the registration of the reference shape to the anatomical reference frame and the spatial relationship between the first portion and the second portion of the flexible elongate device.
